(19) United States
(12) Patent Application Publication
(10) Pub. No.: US 2018/0303351 A1
(43) Pub. Date: Oct. 25, 2018

(54) SYSTEMS AND METHODS FOR OPTIMIZING PHOTOPLETHYSMOGRAPH DATA

(71) Applicants: General Electric Company, Schenectady, NY (US); Board of Trustees of Michigan State University, East Lansing, MI (US)

(72) Inventors: Lalit Keshav Mestha, Cohoes, NY (US); Gayathri Seenumani, Niskayuna, NY (US); Pengfei Meng, Troy, NY (US); Ramakrishna Mukkamala, Okemos, MI (US)

(21) Appl. No.: 15/492,889

(22) Filed: Apr. 20, 2017

(51) Int. Cl. A61B 5/0205; A61B 5/00; A61B 5/1455

(52) U.S. Cl. CPC A61B 5/0205; A61B 5/742; A61B 5/021; A61B 5/7203; A61B 5/6898; A61B 5/14551

(57) ABSTRACT

A system includes an imaging device that captures multichannel image data from a region of interest on a patient, one or more processors, and memory storing instructions. The memory storing instructions cause the one or more processors to receive the multichannel image data from the imaging device, such that the multichannel image data includes an image signal representative of plethysmographic waveform data for the region of interest and specular noise in the multichannel image data. Furthermore, the memory storing instructions cause the one or more processors to generate a projection matrix associated with the multichannel image data and iterate values of the projection matrix to remove the specular noise to generate a representative physiological signal, such that the representative physiological signal has an improved signal-to-noise ratio relative to the image signal and the representative physiological signal is a representative plethysmographic waveform. The memory storing instructions cause the one or more processors to also calculate one or more physiological parameters using the representative physiological signal and output the one or more physiological parameters on a display.

### STATEMENT REGARDING FEDERALLY SPONSORED RESEARCH & DEVELOPMENT

This disclosure was made with Government support under contract number U01EB018818 awarded by the National Institute of Biomedical Imaging and Bioengineering of National Institute of Health. The Government has certain rights in the disclosure.

### BACKGROUND

The subject matter disclosed herein relates to systems and methods for determining physiological parameters using image data received from an imaging device.

Clinicians are interested in monitoring various physiological parameters of a patient that provide information about a patient's health or condition. For example, such parameters may include blood pressure, heart rate, etc. Certain monitoring techniques may involve applying a sensor to a patient's skin and collecting the sensor data to determine the physiological parameter. Contact devices used for monitoring physiological parameters for a prolonged duration may increase the risk of infections or hospital acquired pressure ulcers (HAPUs) in critically ill patients, in particular infants. Sensitive skin, tissue compression, vascular insufficiency to the region, emotional suffering, discomfort, irritation, soreness etc., may be reasons to avoid wearing a contact-based sensor. In addition, wearable sensors may limit mobility of an active patient. For long period of observation/monitoring, a non-contact system that is accurate may be preferred.

### BRIEF DESCRIPTION

In one embodiment, a system includes an imaging device that captures multichannel image data from a region of interest on a patient, one or more processors, and memory storing instructions. The memory storing instructions cause the one or more processors to receive the multichannel image data from the imaging device, such that the multichannel image data includes an image signal representative of plethysmographic waveform data for the region of interest and specular noise in the multichannel image data. Furthermore, the memory storing instructions cause the one or more processors to generate a projection matrix associated with the multichannel image data and iterate values of the projection matrix to remove the specular noise to generate a representative physiological signal, such that the representative physiological signal has an improved signal-to-noise ratio relative to the image signal and the representative physiological signal is a representative plethysmographic waveform. The memory storing instructions cause the one or more processors to also calculate one or more physiological parameters using the representative physiological signal and output the one or more physiological parameters on a display.

In a further embodiment, a method includes acquiring multichannel image data using an imaging device from a region of interest on a patient, such that the multichannel image data includes an image signal representative of plethysmographic waveform data for the region of interest and specular noise in the multichannel image data, such that the multichannel image data includes intensity data, specular data, and pulse data. Further, the method includes normalizing one or more multichannels in the multichannel image data, such that normalizing the one or more multichannels eliminates mean and higher order variations in the intensity data, the specular data, and the pulse data. The method also includes generating a projection matrix of the multichannel image data, iterating values of the projection matrix to remove the specular noise to generate a representative physiological signal, such that the representative physiological signal has an improved signal-to-noise ratio relative to the image signal and the representative physiological signal is a representative plethysmographic waveform. The method further includes calculating one or more physiological parameters using the representative physiological signal and displaying the one or more physiological parameters.

In an additional embodiment, a personal mobile device system includes an imaging device that captures image data over time from a region of interest on a patient, such that the image data includes an image signal representative of plethysmographic waveform data for the region of interest and noise, one or more processors, and a memory storing instructions, such that the instructions cause the one or more processors to normalize color channels in the image data. Color channels are described for this purpose as spectral channels or multiple channels, such that normalizing the color channels includes spatially averaging and temporally averaging the image data. The instructions also cause the one or more processors to generate a projection matrix of the image data, such that the projection matrix is based on a number of spectral components in the image data, iterate values of the projection matrix to remove the noise representative of the specular reflection to generate a representative physiological signal, such that the representative physiological signal has an improved signal-to-noise ratio relative to the image signal and such that the representative physiological signal is a first representative plethysmographic waveform. The instructions also cause the one or more processors to fit a second representative physiological signal to the representative physiological signal, such that the second representative physiological signal is generated based on a model of skin characteristics of the patient, and display the one or more physiological parameters.

### BRIEF DESCRIPTION OF THE DRAWINGS

These and other features, aspects, and advantages of the present disclosure will become better understood when the following detailed description is read with reference to the accompanying drawings in which like characters represent like parts throughout the drawings, wherein:

FIG. 1 is a schematic illustration embodiment of a camera device configured to implement a contactless video-based monitoring system to acquire data indicative of skin characteristics and process the data, in accordance with an aspect of the present disclosure;

FIG. 2 is a schematic illustration of an embodiment of the camera device displaying data indicative of a pixel with respect to wavelength of the video stream, in accordance with an aspect of the present disclosure;

FIG. 3 is a flow diagram depicting an embodiment of a process whereby one or more algorithms are executed to generate optimized parameters, in accordance with an aspect of the present disclosure;

FIG. 4 depicts a two layer skin model for which multichannel RGB data is retrieved, in accordance with aspects of the present disclosure;

FIG. 5 is a flow diagram depicting an embodiment of a specular rejection process, whereby the signal-to-noise ratio (SNR) of the multichannel RGB data is improved, in accordance with aspects of the present disclosure;

FIG. 6 is a flow diagram depicting an embodiment of a process executing a model inversion method, whereby physiological parameters are generated, in accordance with an aspect of the present disclosure;

FIG. 7 depicts an embodiment of a process executing a first stage of the model inversion method of FIG. 6, whereby the error is reduced between averaged values of the RGB from the camera device and RGB values for a skin and camera model, in accordance with aspects of the present disclosure;

FIG. 8 depicts an embodiment of a process executing a second stage of the model inversion method of FIG. 6, whereby the error is reduced between the pulse signal with minimum SNR during real-time or near real-time measurements and the pulse signal extracted from the skin and camera model to generate final skin characteristics, in accordance with aspects of the present disclosure;

FIG. 9 is a schematic diagram depicting an embodiment of a process, whereby skin characteristics and physiological parameters are generated based on a video stream captured by a camera device, in accordance with aspects of the present disclosure;

FIG. 10 depicts results of experimental data comparing SNR between video streams, in accordance with aspects of the present disclosure;

FIG. 11 depicts results of data comparison based on the experimental data of FIG. 10, in accordance with aspects of the present disclosure

FIG. 12 depicts a signal retrieved from scaled skin characteristics from a subject, utilizing the MaxSNR method of FIG. 5, in accordance with aspects of the present disclosure; and

FIG. 13 depicts an embodiment of evaluated correlation of time averaged blood concentration parameter to systolic blood pressure (SBP) and diastolic blood pressure (DBP), in accordance with aspects of the present disclosure.

### DETAILED DESCRIPTION

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions may be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

While the following discussion is generally provided in the context of monitoring physiological parameters (e.g., systolic blood pressure, diastolic blood pressure, pulse rate, etc.) in patients, it should be appreciated that the present techniques are not limited to such medical contexts. Indeed, the provision of examples and explanations in such a medical context is only to facilitate explanation by providing instances of real-world implementations and applications. The present approaches may also be utilized in other contexts, such as the non-invasive inspection of body measurements for animals, and/or the monitoring of athletes, monitoring of drivers or pilots, and so forth.

In particular, the present approach relates to extracting blood volume changes in the skin as applied to humans using red, green, and blue (RGB) cameras, multispectral cameras hyperspectral cameras, and/or any other suitable camera as an alternative to conventional contact-based plethysmograms by using a contactless video-based monitoring system. The above-mentioned cameras may be able to capture multichannel image data, such that the multichannels may include red, green, blue, multispectral, or hyperspectral channels. More specifically, skin characteristics may be optically obtained via a photoplethysmograph (PPG) device. By using RGB, multispectral cameras or hyperspectral cameras, a pulse signal (e.g., representative physiological signal) may be retained from diffused components resulting from the light scattered through the blood flow through the dermis layer of skin and the deeper arteries via a non-invasive method. In this manner, the comfort, convenience, and/or reliability of obtaining certain physiological parameters may be increased for patients being observed for long periods of times. That is, in some instances, by using video taken by a camera, physiological parameters may be comfortably, conveniently, and/or reliably obtained. Further, the present approach has potential for application for remote healthcare for episodic continuous monitoring at homes, clinics in rural villages, locations that may be far from specialists, etc.

The present approach extracts physiological parameters from skin characteristics of an optical model that reduces the effects of light intensity variations and specular light reflections to improve (e.g., maximize) the signal-to-noise ratio (SNR). That is, a MaxSNR method includes solving a constrained optimization problem to mitigate the effects of motion, variations in camera, lighting, and skin tone to lead to a suitable separation between the pulse, specular, and/or intensity components of the captured image, as discussed in detail below.

In addition, the proposed approach uses the pulse signal (e.g., representative physiological signal) with the improved SNR obtained according to the techniques provided herein to extract physiological parameters (e.g., pulsating blood concentration parameters, blood oxygen saturation, heart rate variability, heart rate, blood pressure, etc.,) by inverting a parameterized optical model of the human skin. That is, a model inversion method is used to predict certain skin characteristics (e.g., effective values of melanin concentration, thickness of the epidermis layer, blood volume concentration, oxygen saturation, spectral scattering, etc.) that produce the multichannel (e.g., RGB) signals from a nonlinear skin model generated for a certain skin characteristic setting. In this manner, signal variability that is unrelated to the underling physiological parameter can be removed or accounted for.

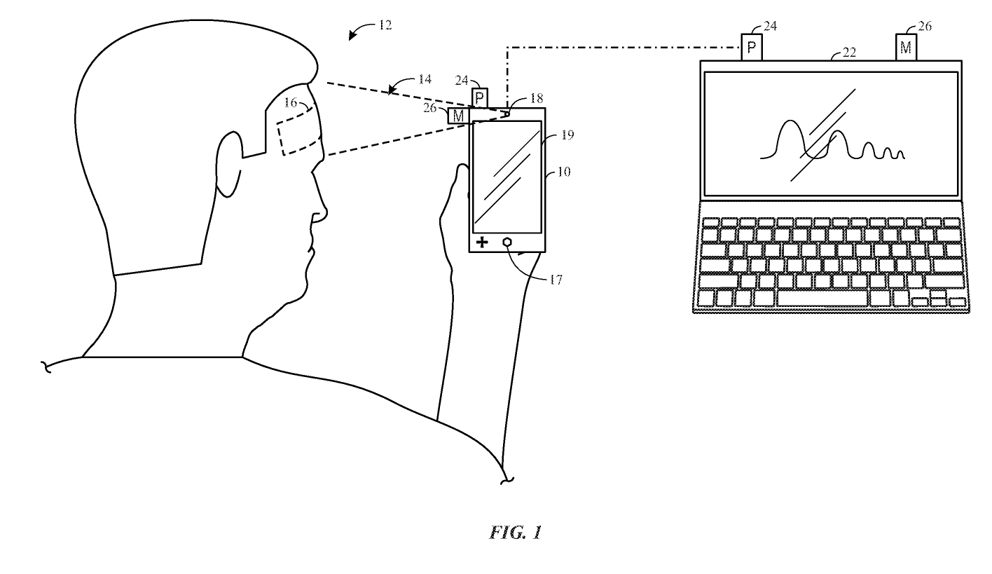

With the foregoing in mind, FIG. 1 is a schematic illustration embodiment of a computing device configured to implement a contactless video-based monitoring system to assess physiological parameters. As illustrated, a user 12 (e.g., hospital patient) may capture a video stream 14 of their forehead 16 using a camera device 10 to extract physiological parameters. While the illustrated embodiment shows the user 12 acquiring video stream 14 of the user's own forehead, in some instances, the video stream 14 may also be taken from any substantially exposed body surface rich in blood vessels (e.g., cheek, back of hand, etc.).

In some embodiments, the camera device 10 may be a personal mobile device (e.g., cellular device, laptop, tablet, etc.) that may include a camera 18 that may record video stream 14 of the environment presented before the camera 18. The camera 18 may include complementary metal-oxide-semiconductor (CMOS) image sensors, a charge-coupled device (CCD) camera, any multispectral camera, any hyperspectral camera, any multichannel camera such as a 3-channel RGB camera, etc. Furthermore, the disclosed subject matter may be implemented by the personal mobile device. It should be noted, that the disclosed subject matter may help correct anomalies that may arise due to camera differences. That is, the disclosed embodiments account for variations in image data that are the result of camera quality or configuration. By provided improved techniques for removing noise (i.e., acquired data that does not relate to the physiological parameter), such as camera or ambient light-related artifacts, the disclosed techniques may be used in conjunction with a variety of camera types and in a variety of lighting environments.

As illustrated, the camera device 10 may include user input buttons 17 that may help in the selection and navigation of options displayed on the graphical user interface (GUI) of the camera device 10. Furthermore, as illustrated, the camera device 10 may include a display 19 that may show the GUI of the camera device 10 and allow the user to navigate the GUI and make selections (e.g., to take video stream 14, power on the camera device 10, export data, etc.). In some instances, the camera device 10 may receive user inputs via the display 19 (e.g., via a touch-screen configuration) to, for example, acquire the video stream 14. In other instances, the camera device may receive user inputs via a combination of inputs to the buttons 17 and tactile inputs to the display 19.

In some embodiments, the camera device 10 may be communicatively coupled to an external network or external computing device 22 (e.g., laptop, desktop, parallel computing system, etc.). For example, the camera device 10 may couple to a network, such as a personal area network (PAN), a local area network (LAN), or a wide area network (WAN). In some embodiments, the camera device 10 may be communicatively coupled to the computing device via a wireless or landline connection to, for example, receive and transmit data 20. Accordingly, in some embodiments, the camera device 10 may export the video stream 14 or any other data 20 to an external computing device 22 for further processing. Furthermore, the camera device 10 may also receive data 20 back from the external computing device 22 to, for example, display results on display 19. In other embodiments, the camera device 10 may process the acquired video stream 14 via an application operating on the camera device 10. The application may process the acquired video stream 14 locally and/or may also communicate with the external computing device 22 as part of the processing.

In the depicted embodiment, the external computing device 22 includes a processor 24 that may execute instructions stored in memory 26 to perform operations, such as determine physiological parameters. In some instances, the processor 24 may include one or more general purpose microprocessors, one or more application specific processors (ASICs), one or more field programmable logic arrays (FPGAs), or any combination thereof. Additionally, the memory 26 may be a tangible, non-transitory, computer-readable medium that store instructions executable by and data to be processed by the processor 24. For example, in the depicted embodiment, the memory 26 may store algorithms that execute and calculate the subject matter discussed below. Thus, in some embodiments, the memory 26 may include random access memory (RAM), read only memory (ROM), rewritable non-volatile memory, flash memory, hard drives, optical discs, and the like.

It should be noted that, in some embodiments, the camera device 10 may be a standalone device (e.g., that does not require the aid of an external computing device 22) and may include the processor 24 and memory 26 to execute the subject matter discussed in detail below. That is, in some embodiments, the camera device may execute the subject matter below via an internal processor 24 that may execute instructions stored in memory 26 to, for example, determine physiological parameters after obtaining a video stream 14 (e.g., of a forehead).

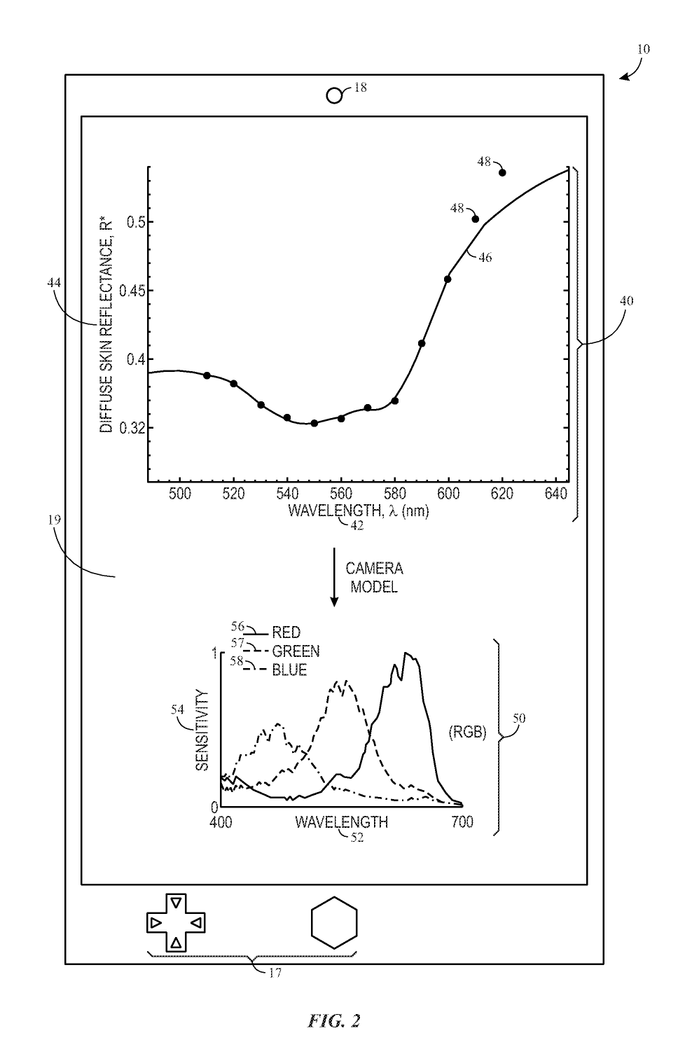

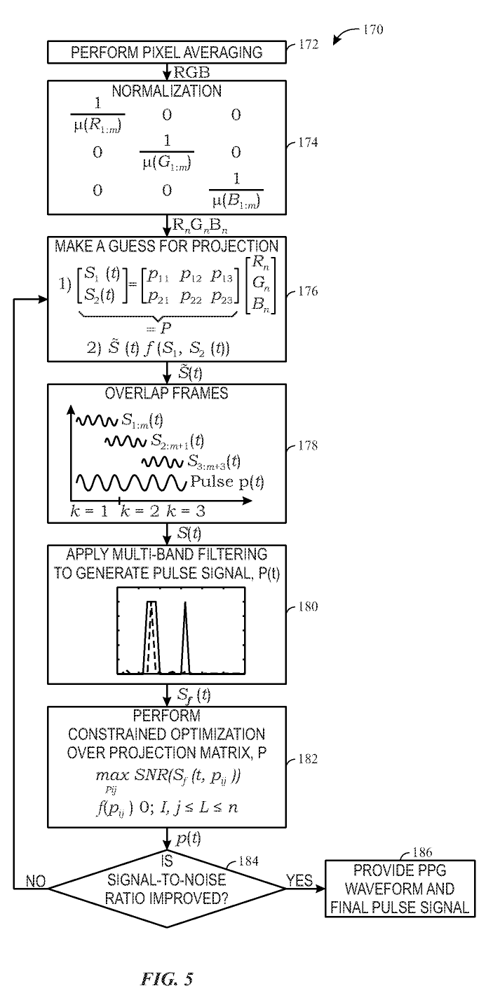

Turning to FIG. 2, included is a schematic illustration of an embodiments of the camera device 10 displaying data 20 (e.g., on display 19) for one pixel indicative of the video stream 14. It is to be noted that, for illustration, this description is shown for a spectral image pixel captured by a hyperspectral imager. However, in further embodiments, any of the above-mentioned image capturing devices (e.g., cameras) may be used. After the camera 18 captures video steam 14 of a substantially flat surface (e.g., forehead 16), the processor 24 may execute the calculations discussed below with regards to FIG. 4 to determine a reflectance spectra 40 for a two layer skin model. More specifically, after capturing video stream 14, for each pixel captured over time, the processor 24 may determine the diffuse skin reflectance 44, R*, corresponding to each wavelength 42, λ, in nanometers (nm). As illustrated, the processor 24 may generate a plot of the diffuse skin reflectance 44, R* vs. the wavelength 42, λ, similar to that displayed for the reflectance spectra 40.

Furthermore, the processor 24 may take the data indicative of the reflectance spectra 40 and multichannel image data, hereinafter also called “RGB image data 50 that plots the sensitivity corresponding to each wavelength for the colors red, green, and blue, based on their respective filters (e.g., red filter, green filter, blue filter, etc.), which may be obtained from manufacturer's data. Although the present approach includes a discussion of using RGB image data, it should be noted that any multichannel image data may be used. As illustrated, the display 19 may display the illustrated plot, which may include a line graph 56 corresponding to red, a line graph 57 corresponding to green, and a line graph 58 corresponding to blue.

In some embodiments, the processor 24 may calculate and store in memory 26 RGB values over time (e.g., the time duration of the video stream 14). In some instances, the processor 24 may perform the calculations discussed below with regards to FIG. 4 at a certain time stamp. For example, RGB image data 50 may be determined for any time stamp interval, such as every 10 milliseconds (ms), 100 ms, 1 second, or any suitable time stamp interval.

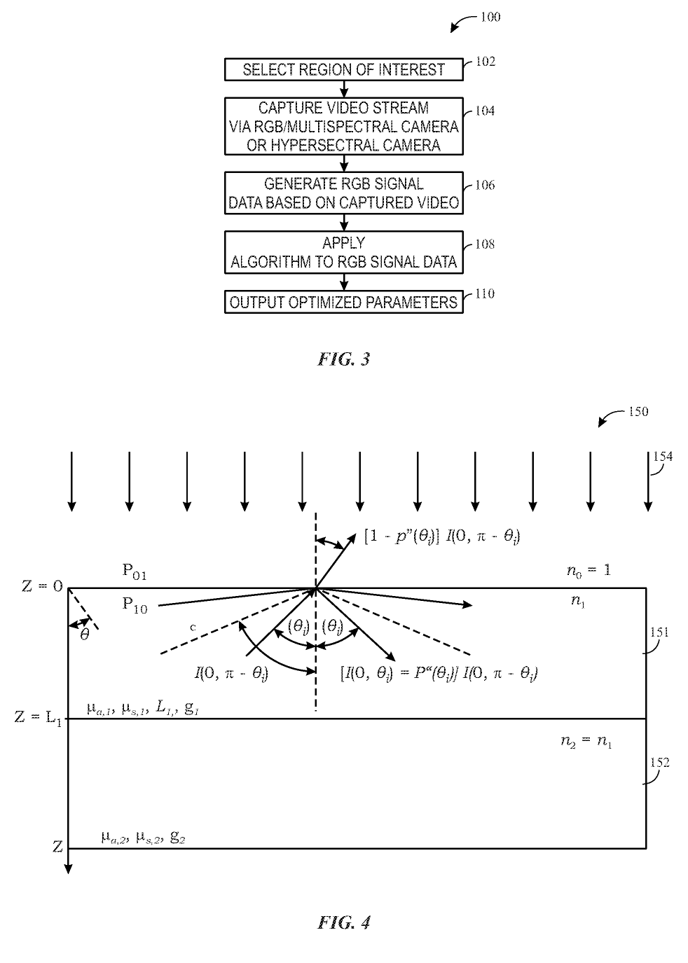

FIG. 3 is a flow diagram 100 illustrating an embodiment of a process whereby one or more algorithms are executed by processor 24 of the camera device 10 to generate optimized parameters. More specifically, the camera device 10 selects a region of interest to capture the video stream 14. After capturing the video stream 14, the processor 24 may generate RGB image data 50 based on the captured vide stream, such that the captured video stream may provide RGB image data 50 indicative of each pixel over a time interval. In some embodiments, the RGB image data 50 may include an image signal representative of plethysmographic waveform data for the region of interest and specular noise in the RGB image data. The processor 24 may apply algorithms to the RGB image data 50 to determine RGB image data with a maximum SNR and/or predict physiological parameters as discussed in detail below.

With regards to selecting a region of interest (process block 102), in some embodiments, the camera device 10 may scan the surface (e.g., of skin) reflecting light back to the lens of the camera. In some instances, the camera device 10 may scan a surface within a distance range away from the camera device and facing the camera device 10. For example, the camera device may scan a surface between 0.1 meters (m) and 1 m, or any other suitable distance.

In some instances, after scanning the surface in front of its lens, the camera device 10 may select a substantially flat surface as the region of interest. In some embodiments, selecting the substantially flat surface may include excluding any surfaces not substantially orthogonally oriented (e.g., between 75 and 105 degrees) towards the lens of the camera.

After selecting the region of interest (process block 102), the camera device 10 may capture the video stream 14 (process block 104). The above-mentioned camera device may be any imaging device able to capture multichannel image data, such that the multichannels may include red, green, blue, multispectral, or hyperspectral channels. In some instances, the camera device may capture video stream 14 of the region of interest (e.g., a substantially flat surface of the skin) that may include information indicative of the pixels captured in the video stream. For example, the camera device may capture the video stream 14 for any length of time (e.g., 500 ms, 1 sec, 5 sec, or any suitable length of time). Furthermore, the camera device 10 may capture and store in memory 26 a time, coordinates (e.g., x, y, z coordinates), and other suitable information corresponding to each pixel captured by the camera device 10.

The processor 24 of the camera device 10 generates multichannel image data 50 based on the video stream 14 captured by the camera device (process block 106). In some embodiments, generating RGB image data 50 may include separating the light received by the camera device into the three RGB primary colors by using prisms, filters, and/or video camera tubes. In some instances, a charge-coupled device (CCD) image sensor may enhance the detection of light and separation of the light into the three RGB primary colors. Furthermore, generating RGB image data may include using a Bayer filter arrangement to interpolate data via various channels to compile RGB image data 50 for the region of interest captured by the camera device 10. It should be noted that the RGB image data may be generated for the duration of the video stream for the captured region of interest. The RGB image data 50 may be stored in memory 24 for further processing.

That is, one or more algorithms are applied to the RGB image data (process block 108). As mentioned above and described in detail below with regards to FIGS. 4-9, the RGB image data is projected to certain directions that are computed via optimization based MaxSNR method to improve the SNR to mitigate the effects of motion variations in camera, lighting, skin tone, etc. The multichannel (e.g., RGB) image data with the maximum SNR is used to solve an inverse problem, whereby the skin characteristics of equation 1 of FIG. 4 are predicted.

After determining the maximum SNR for the RGB signal data and/or predicting skin characteristics by applying one or more algorithms to the RGB image data 50, the processor may output relevant optimized parameters (process block 110). In some embodiments, outputting the optimized parameters may include displaying on display 19 of the camera device 10 the optimized RGB image data with the maximum SNR determined by the MaxSNR method described in detail with regards to FIGS. 5 and 6. In some embodiments, outputting the optimized parameters may include displaying the optimized predicted skin characteristics on the display 19.

For context with regards to some calculations that may be performed by a processor 24, FIG. 4 includes a schematic illustration of an embodiment of a two layer model of the skin 150 exposed to light 154 that bounces back to the lens of the camera device 10 to compile the video stream 14. As illustrated, the model of the skin 150 includes a first layer, hereinafter called the “epidermis 151,” and a second layer, hereinafter referred to as the “dermis 152.” The epidermis 151 may have a thickness of L₁ between 20 and 150 micrometers (μm). The calculations described below may be performed by the processor 24 of the camera device 10 to calculate a skin parameter vector, defined as:

p=└LₑₚᵢCₘₑₗƒ_{blood}SO₂Cₛ┘ (1)

such that Lₑₚᵢ is the thickness of the epidermis 151, Cₘₑₗ is the melanin concentration, ƒ_{blood} is defined as the volume fraction of the dermis occupied by blood, SO₂ is the blood oxygen saturation, and Cₛ is the scattering coefficient in both the epidermis 151 and dermis 152. In some embodiments, the skin parameter vector may help determine skin characteristics.

### Two Layer Spectral Skin Model

In more detail, the mathematical equations discussed below establish relationships between the reflectance of light for a two layer skin model. The semi-empirical two layer reflectance, R₌, is defined in equation 2 as:

R₌=R*R₋(wₜᵣ₁)+(1−R*)R₋(wₜᵣ₂) (2)

R₋ is the diffuse reflectance obtained from the Kubelka-Munk model for semi-infinite medium (e.g., single layer solutions) defined in equation 3 as:

$${R}_{-} ( {w}_{tr} ) = [ 1 - {ρ}_{01} ] [ 1 - {\hat{ρ}}_{10} ( {w}_{tr} ) ] \frac{{\hat{R}}_{d} ( {w}_{tr} )}{1 - {\hat{ρ}}_{10} ( {w}_{tr} ) {\hat{R}}_{d} ( {w}_{tr} )} , \qquad ( 3 )$$

R* is the reduced reflectance defined in equation 4 as:

$${R}^{*} = \frac{tanh ( {Y}_{1} )}{\frac{1}{α} + ( 1 - \frac{1}{α} ) tanh ( {Y}_{1} )} \\ and \frac{1}{α} = C ( {n}_{1} ) {w}_{tr 2}^{2} + D ( {n}_{1} ) {w}_{tr 2} + E ( {n}_{1} ) \\ For {n}_{1} = 1.44 ; \\ C ( {n}_{1} ) = - 0.569 , D ( {n}_{1} ) = - 0.055 , E ( {n}_{1} ) = 0.993 , \qquad ( 4 )$$

wₜᵣ₁ is the scattering albedo for the first layer 151 defined in equation 5 as:

wₜᵣ₁(λ)=μ_{s,tr}(λ)/[μ_{a,epi}(λ)+μ_{s,tr}(λ)], (5)

wₜᵣ₂ is the scattering albedo for the second layer 152 defined in equation 6 as:

wₜᵣ₂(λ)=μ_{s,tr}(λ)/[μ_{a,derm}(λ)+μ_{s,tr}(λ)], (6)

The reflectivity, \hat{ρ}₁₀(wₜᵣ), is defined in equation 7 as:

$${\hat{ρ}}_{10} ( {w}_{tr} ) = {ρ}_{10} + {\sum }_{i = 0}^{i = N} {{A}_{i} [ a ( {w}_{tr} ) ]}^{i} \\ {ρ}_{01} = {( \frac{{n}_{1} - {n}_{0}}{{n}_{1} + {n}_{0}} )}^{2} \\ {n}_{1} = {n}_{2} = 1.44 ; refractive \\ indicies of layers 1 \& 2 \qquad ( 7 )$$

The diffuse reflectance, \hat{R}_{d}(wₜᵣ) is defined in equation 8 as:

$${\hat{R}}_{d} ( {w}_{tr} ) = {\tilde{R}}_{d} ( a ( {w}_{tr} ) ) + {\sum }_{i = 0}^{i = N} {{B}_{i} [ a ( {w}_{tr} ) ]}^{i} \qquad ( 8 )$$

such that {Aᵢ, Bᵢ} are regression coefficients of N polynomial order, and a(wₜᵣ) are found from the Kubelka-Munk equation.

### Model of Scattering

The scattering spectra for the first layer 151 and second layer 152 are assumed to be similar and defined in equation 9 as:

$${μ}_{s , tr} ( λ ) = {{C}_{s} ( \frac{λ}{{λ}_{0}} )}^{- b} \qquad ( 9 )$$

where Cₛ is a constant between the range of 10⁵ and 10⁶ cm⁻¹, b=1.3 and represents the average size of the connective tissue responsible for the scattering, and λ₀=1.

### Model of Epidermis Layer

The absorption spectra for the epidermis may be defined in equation 10 as:

μ_{a,epi}(λ)=μ_{a,mel}(λ)ƒₘₑₗ+μ_{a,back}(λ)(1−ƒₘₑₗ) (10)

such that ƒₘₑₗ is the melanin concentration (e.g., in mg/mL), typically within the range of 0-100 mg/mL, the absorption coefficient of melanosomes is defined as μ_{a,mel}(λ)=6.60×10¹¹λ^{-3.33}, the background absorption of human flesh is defined as μ_{a,back}(λ) 7.81×10⁸λ^{-3.255}, such that λ is in nanometers (nm) μ_{a,mel}(λ) and μ_{a,back}(λ) is in cm⁻¹.

### Model of Dermis Layer

The absorption spectra for the dermis is in cm′ may be defined in equation 11 as:

μ_{a,derm}(λ)=ƒ_{blood}μ_{a,blood}(λ)+(1−ƒ_{blood})μ_{a,back}(λ) (11)

such that the volume fraction of the dermis occupied by blood ƒ_{blood}, typically ranges from 0.2 to 7%.

Further, the absorption coefficient of blood, μ_{a,blood} is a function of the blood oxygen saturation, SO₂, and may be defined in equation 12 as:

μ_{a,blood}(λ)=μ_{a,oxy}(λ)+μ_{a,deoxy}(λ) (12)

such that

μ_{a,oxy}(λ)=SO₂Cₕₑₘₑε_{oxy}(λ)/66,500 (13)

μ_{a,deoxy}(λ)=(1−SO₂)Cₕₑₘₑε_{deoxy}(λ)/66,500 (14)

for hemoglobin concentration in blood, Cₕₑₘₑ=150 g/L, and extinction coefficients of deoxygenated (Hb) hemoglobin, ε_{oxy}, and oxygenated (HbO₂) hemoglobin, ε_{deoxy}, where f_{blood} is the volume fraction of the dermis occupied by blood, typically ranging from 0.2% to 7%, and where the absorption coefficient of blood is defined in equation 15 as:

μ_{a,blood}(λ)=μ_{a,oxy}(λ)+μ_{a,deoxy}(λ) (15)

μ_{a,oxy}(λ)=SO₂Cₕₑₘₑε_{oxy}(λ)/66,500 (16)

After the semi-empirical two layer reflectance, R₌, is determined for the pixels captured in the region of interest using equation 2 and the above referenced equations, the processor 24 performs the process depicted in FIG. 5 as part of applying an algorithm to the RGB image data. That is, FIG. 5 is a flow diagram 170 depicting an embodiment of a process executing the MaxSNR method, whereby the signal-to-noise ratio (SNR) of the RGB (e.g., time-varying) data is improved. The MaxSNR method depicted as a process in flow diagram 170 develops on the idea that there are optimal, non-constant, combinations of chrominance signals which have greater pulse-specular separation. In other words, the disclosed subject matter targets at extracting pulsatile signals with SNR that is as close as possible to PPG signals which have better robustness properties. More specifically, flow diagram 170 proceeds by performing pixel averaging for the pixels included in the region of interest determined by the camera device 10. Then, the RGB image data is normalized. That is, the color channels corresponding to the RGB image data are normalized. After normalizing the RGB image data, an initial projection direction is predicted. The pulse signal (e.g., representative physiological signal) and SNR associated with the initial projection are computed and the optimal pulse signal is determined by applying constrained optimization to the video frames of the video stream 14. The optimization iteratively updates the projection directions, starting with the initial guess, by considering variations of the SNR in that direction. The iterates proceed until no further improvement in SNR can be obtained. The pulse signal associated with the projection matrix of the final iterate yields the optimal SNR and (e.g., representative physiological signal) is provided as final output and the PPG waveform is generated for the video sequence.

In more detail, pixel averaging is performed by the processor 24 (process block 172). In some embodiments, the pixel averaging may include both spatial averaging and temporal averaging. In other embodiments, the pixel averaging may only include one of either spatial averaging or temporal averaging of the RGB image data. In some embodiments, the RGB image data may include an image signal representative of plethysmographic waveform data for the region of interest and specular noise in the RGB image data. In some embodiments, the R(t), G(t), and B(t) signals are translated into intensity, i(t), specular s(t), and pulse, p(t) as shown in equation 17:

$$vPPG ( t ) = [ \begin{matrix}R ( t ) \\ G ( t ) \\ B ( t )\end{matrix} ] = ( {I}_{nom} + i ( t ) ) ( c + [ \begin{matrix}{R}_{p} \\ {G}_{p} \\ {B}_{p}\end{matrix} ] p ( t ) + [ \begin{matrix}{R}_{s} \\ {G}_{s} \\ {B}_{s}\end{matrix} ] s ( t ) ) \qquad ( 17 )$$

where the intensity, specular and pulse signals can be represented as a constant and time varying components. It must be noted that the time-varying intensity components are due to the changes in relative motion between source and subject and are less in amplitude. It should be noted that, the vector p of equation 1 is different from the pulse, p(t) in equation 17.

After the processor performs pixel averaging, the processor normalizes the RGB data (process block 174) that has been averaged. In some instances, normalizing the RGB values gives the RGB values whose numeric values will range between zero and one and may mitigate the effects of quantization noise, motion, etc. Normalizing the pixels may include normalizing the RGB values using equation 18, as shown below:

$${PPG}_{norm} ( t ) = N = [ \begin{matrix}\frac{1}{μ ( {R}_{1 : m} )} & 0 & 0 \\ 0 & \frac{1}{μ ( {G}_{1 : m} )} & 0 \\ 0 & 0 & \frac{1}{μ ( {B}_{1 : m} )}\end{matrix} ] \qquad ( 18 )$$

In some embodiments normalizing the data may include performing the calculations of equation 19, thereby eliminating the mean and higher order variations in intensity, pulse, and specular components.

$${vPPG}_{norm} = NvPPG ( t ) = 1 ( 1 + i ( t ) ) + {NI}_{nom} ( \underbrace{[ \begin{matrix}{R}_{p} \\ {G}_{p} \\ {B}_{p}\end{matrix} ]}_{{v}_{p}} p ( t ) + \underbrace{[ \begin{matrix}{R}_{s} \\ {G}_{s} \\ {B}_{s}\end{matrix} ]}_{{v}_{s}} s ( t ) ) , \\ where NE ( vPPG ) = 1 \qquad ( 19 )$$

After normalizing the pixels and generating a normalized diagonal matrix, the projection matrix P is predicted (process block 176). That is, the projection matrix is chosen to be a matrix that may generate the maximum SNR possible for the RGB values obtained via the camera device 10 over time. In certain instances, choosing the projection matrix, P, may include choosing P, such that the intensity variations may be eliminated. In some instances, choosing the projection matrix P, may include choosing P, such that S(t)=f(P.vPPGₙₒᵣₘ) has a maximum

$$\frac{p ( t )}{s ( t )}$$

or lower S(t), such that S(t) is defined in equation 20 as:

$$[ \begin{matrix}{S}_{1} ( t ) \\ … \\ {S}_{n} ( t )\end{matrix} ] = P {vPPG}_{norm} ( t ) \qquad ( 20 )$$

where n is the number of spectral components in the video, which in this example is n=3 for each of the three colors corresponding to the RGB image data 50.

For example, for S(t) with two components, the calculations would be performed in accordance with equations 21 and 22.

$$\begin{matrix}[ \begin{matrix}{S}_{1} ( t ) \\ {S}_{2} ( t )\end{matrix} ] = [ \begin{matrix}{p}_{11} & {p}_{12} & {p}_{13} \\ {p}_{21} & {p}_{22} & {p}_{23}\end{matrix} ] [ \begin{matrix}{R}_{n} \\ {G}_{n} \\ {B}_{n}\end{matrix} ] = P {vPPG}_{norm} & ( 21 ) \\ \overline{S} ( t ) = f ( {S}_{1} , {S}_{2} ( t ) ) & ( 22 )\end{matrix}$$

Furthermore, after determining a projection, frames are overlapped (process block 178) to prepare the specular values to generate a pulse signal p(t) (e.g., representative physiological signal). More specifically, the normalized RGB data, VPPGₙₒᵣₘ(t), is multiplied with the predicted projection matrix, P, to produce a signal in accordance with equation 20. The pulse signal, p(t), may be extracted from the projection direction and S(t) via equation 24 after determining S(t) via equation 23, which may be defined as:

S(t)=ƒ(S₁(t), . . . ,Sₙ(t)),ƒ: [symbol] ̂n→ [symbol] (23)

After determining S(t) via equation 23, S(t) is filtered using a multi-band filter (process block 180) to construct a filtered specular values, S_{ƒ}(t). In some embodiments the filter represents the physiological components (e.g., fundamental at the pulse rate frequency, first harmonic, second harmonic, etc.). Furthermore, the pulse signal, p(t), may be determined (process block 180) by computing S_{ƒ}(t) with overlapping batches (e.g., 50 to 100 frame overlaps) via equation 24.

pulse(κ)=pulse(κ)+S_{ƒ}(κ)−E[S_{ƒ}(κ)],κ∈t:t+M,M∈[50,10] (24)

Afterwards, the constrained optimization is solved over projection matrix P (process block 182) for a frame length given by utilizing equation 25. It should be noted that the SNR is computed based on the multi-band filtering of the pulse signal (e.g., representative physiological signal).

$${max}_{{p}_{ij}} SNR ( {s}_{f} ( t , {p}_{ij} ) ) \\ f ( {p}_{ij} ) = 0 , i , j \leq L \leq n \qquad ( 25 )$$

In some embodiments, the constraint in equation 25 can represent the orthogonality of the projection matrix to unit vector.

In some embodiments, the projection direction is considered to be a 3×1 vector in the family of unit length vectors. In such cases the optimization variable pᵢⱼ is a scalar x and the vector is given by 26. Here, the optimization solves for parameter x that would improve (e.g., maximize) the SNR of the pulse signal computed in the projected direction P. Such mechanism may be considered when computational time requirements are stringent.

$$P = [ \frac{x}{\sqrt{6}} + \frac{\sqrt{1 - {x}^{2}}}{\sqrt{2}} , \frac{x}{\sqrt{6}} - \frac{\sqrt{1 - {x}^{2}}}{\sqrt{2}} , \frac{- 2 x}{\sqrt{6}} ] \qquad ( 26 )$$

In some embodiments, the pulse signal, p(t), is analyzed by the processor 24 to determine if the SNR has been improved (decision block 184). In some embodiments, this may include identifying if

$$\frac{p ( t )}{s ( t )}$$

is improved, p(t) is improved, or if s(t) has been reduced.

If the SNR is improved (e.g., such that no projection P can increase the SNR), the processor 24 provides the pulse signal, p(t), as the target final signal and produces the PPG waveform (process block 186). In some embodiments, the PPG waveform and/or pulse signal may be displayed on the display 19 of the camera device or computing device 22 after the PPG wave form and final pulse signal have been determined. In some instances, the final pulse signal may include a representative plethysmographic waveform signal.

Alternatively, if the SNR has not been improved (e.g., such that a different projection P may exist), the processor 24 reverts back to making a different choice for projection P (process block 176). In some embodiments, the additional choice for projection P may be based on the SNR generated by the constrained optimization. In this manner, flow diagram 170 (and the MaxSNR method) iteratively performs process steps 176 through 184. In some embodiments, the flow diagram iteratively performs process steps 176 through 184 until the SNR has been improved.

Turning to FIG. 6, illustrated is a flow diagram 200 of a model inversion method whereby final physiological parameters are generated. More specifically the model inversion method begins by using the spatially averaged RGB image data to determine a final pulse signal (e.g., representative physiological signal). Skin characteristics (e.g., melanin concentration, thickness of the epidermis layer, blood volume concentration, oxygen saturation, spectral scattering, etc.) of vector p of equation 1, that may have produced the pulse signal are estimated (e.g., initially guessed and iteratively determined). A scaling factor is applied to the estimates of the skin characteristics. Then, an objective function is used to compute the summation of pulse signal error over a time interval until the pulse signal error is reduced, at which point the final skin characteristics are produced.

In more detail, the model inversion method illustrated in flow diagram 200 receives averaged RGB data, as discussed above in detail with regards to process block 172 of FIG. 5. That is, as discussed above the averaged RGB image data may be determined by the camera device 10 based on the video stream 14. After determining averaged RGB image data, final pulse signal is received (process block 202) by the processor 24. In some embodiments, the final pulse signal may be generated by the MaxSNR method described in detail in FIG. 5.

After receiving the final pulse signal (e.g., via the MaxSNR method), the processor 24 estimates the skin characteristics (process block 203), included in estimate vector p₀ as shown in equation 27:

p₀=[CₘₑₗLₑₚᵢƒ_{blood}SO₂Cₛ]₀ (27)

such that p₀ may produce the averaged RGB image data. In some embodiments, the skin characteristics of the estimated vector p₀ may be determined according to the equations described above with regards to FIG. 4. For example, ƒ_{blood} may be determined by equation 11. In some instances, the skin characteristics of the estimate vector p₀ are guessed by the processor 24 based on the RGB image data.

In some instances, estimating the skin characteristics (process block 203) may include checking to see if the skin characteristics of the estimated vector p₀ produce the skin and camera model with RGB image data that closely resemble to averaged RGB image data retrieved by the camera device 10 based on the video stream 14. That is, the RGB data of a skin and camera model that includes the skin characteristics (e.g., melanin concentration, thickness of the epidermis layer, blood volume concentration, oxygen saturation, spectral scattering, etc.) of the estimated vector, p₀, are compared to the averaged RGB image data 50 from the camera device 10. That is, when the difference between the RGB data of the skin and camera model associated with the skin characteristics of the estimated vector, p₀, and the average RGB image data from the camera 10 is reduced, the pulse signal (e.g., representative physiological signal) associated with the RGB data of the skin and camera model is generated.

In some embodiments, when the RGB data associated with the skin characteristics of the estimated vector p₀ are not close to the averaged RGB image data 50 from the camera device, the processor 24 respectively applies a scaling factors (process block 204) to the respective components of the skin characteristics of the estimated vector. Applying the scaling factors to equation 27 produces a vector of scaled skin characteristics, pₛ, as shown in equation 28:

$${p}_{s} = [ \begin{matrix}\frac{{L}_{epi}}{{α}_{1}} & \frac{{C}_{mel}}{{α}_{2}} & \frac{{f}_{blood}}{{α}_{3}} & \frac{{SO}_{2}}{{α}_{4}} & \frac{{C}_{s}}{{α}_{5}} ]\end{matrix} \qquad ( 28 )$$

such that the scaling factors of the scaling vector, α=[α₁ α₂ α₃ α₄ α₅] are determined based on a Jacobian analysis for the design space of equation 27.

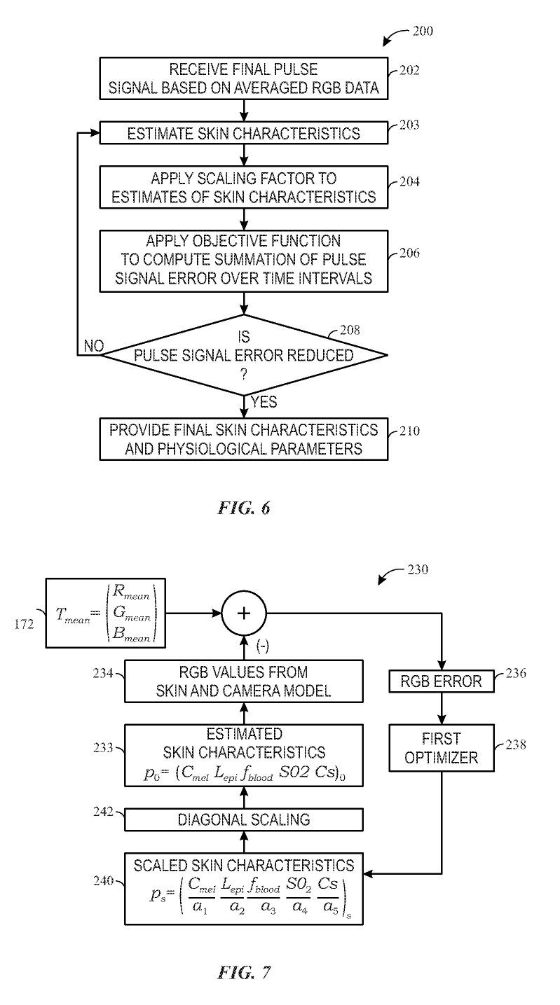

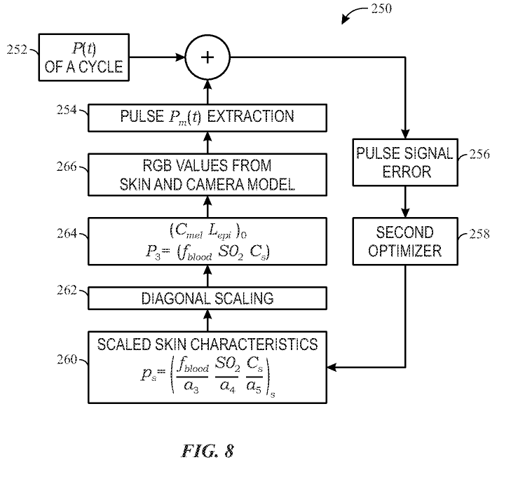

In some embodiments, applying the scaling factor to the estimates of the skin characteristics (e.g., estimate vector p₀) and generating a vector pₛ of scaled skin characteristics, may cause the RGB data associated with pₛ to be compared to the averaged RGB image data from the camera device 10. That is, when the difference between the RGB data of the skin and camera model associated with the skin characteristics of the scaled vector, pₛ, and the average RGB image data from the camera 10 is reduced, the pulse signal associated with the RGB data of the skin and camera model is generated. A flow diagram illustrating this iterative process is provided in the discussion of FIG. 7, below.

The processor applies an objective function to compute the summation of the pulse signal error over time (process block 206). That is, the vector of scaled skin characteristics, pₛ, and its parameters is estimated over time using RGB values corresponding to each frame from the region of interest. In some instances, the time interval of interest may be the entire duration of the video stream 14 captured by the camera device 10. In certain instances, the time interval may be 10 ms, 100 ms, 1 second, 10 seconds, or any other suitable time interval. In some embodiments, the pulse, pₘ(t) corresponding to the skin and camera model may be compared to the pulse signal, p(t) via nonlinear analysis of equation 29:

$$\frac{\partial {f}_{obj}}{\partial x} = {lim}_{h \to 0} \frac{Im ⌊ {f}_{obj} ( x + ih ) ⌋}{h} \qquad ( 29 )$$

where the objective function, ƒ_{obj}, is given by equation 30:

$${f}_{obj} = {\sum }_{t = {t}_{1}}^{{t}_{2}} {( {P}_{m} ( t ) - P ( t ) )}^{2} = {‖ {( {P}_{m} - P )}_{{t}_{1} ∼ {t}_{2}} ‖}^{2} \qquad ( 30 )$$

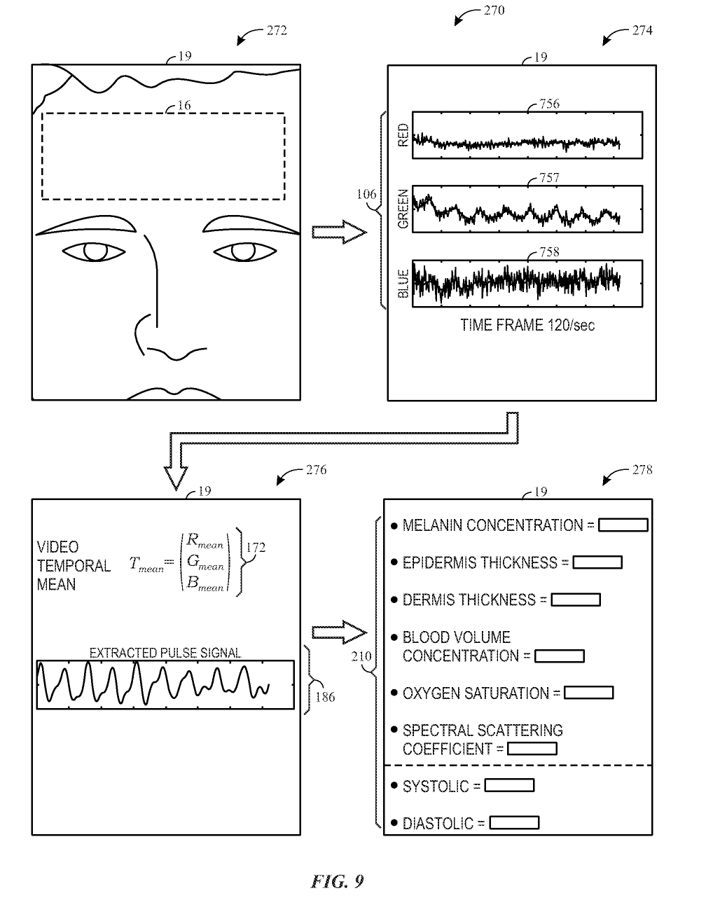

The value computed by the objective function, ƒ_{obj}, is indicative of the pulse signal error. After information indicative of the pulse signal error is generated, the processor determines if the pulse signal is reduced (decision block 208). Since a smaller value for ƒ_{obj} corresponds to a smaller error between the pulse signal generated from the camera device 10, the process of flow diagram 200 iterates between process blocks 203 and 208 until the ƒ_{obj} is reduced. An example of this iterative process is illustrated in FIG. 9. More specifically, the objective function computes the summation of pulse signal error over a time interval. When the pulse signal error over the time interval is not reduced the skin characteristics are estimated (process block 203) and the flow diagram 200 proceeds as described above.

Alternatively, when the pulse signal error of the time interval (e.g., and the objective function) is reduced according to equation 29, the skin characteristics of equation 1 are provided as final. In some embodiments, providing the final skin characteristics may include displaying the skin characteristics (e.g., the values corresponding to the variables of equation 1) on the display 19 of camera device 10.

FIG. 7 depicts an embodiment of a process 230 executing a first stage of the model inversion method of FIG. 6, whereby the error (hereafter called “RGB error”) is reduced between averaged values of the RGB from the camera device and RGB values for a skin and camera model. After the error is reduced, the RGB values corresponding to the skin and camera model (block 234) are stored and used in the second stage of the model inversion method, as described in detail with regards to FIG. 8.

In more detail, the camera device 10 observes RGB channels and the video stream 14 is spatially averaged to generate averaged RGB values, Tₘₑₐₙ=[Rₘₑₐₙ Gₘₑₐₙ Bₘₑₐₙ]^{T} (block 172). From these averaged RGB values, the skin characteristics of equation 1 may be extrapolated. That is, the process 230 estimates the skin characteristics of equation 1 (block 233), as discussed above with regards to equation 27. Based on these estimates of the skin characteristics, a skin and camera model may be developed for those estimates of the skin characteristics, as discussed with regards to equation 27. The RGB values for the skin and camera model are calculated (block 234).

After calculating RGB values for the skin and camera model (block 234) based on estimated parameters (block 233), the RGB error is computed (block 236). After determining the RGB error (e.g., the difference between the values of block 172 and block 234), the RGB error is identified by the first optimizer (block 238). In some instances, if the algorithm of the first optimizer determines that the RGB error is at a minimum, the RGB values and the skin and camera model are stored in memory 26 of the camera device 10. In other words, when the difference between the averaged RGB values from the video stream 14 and the RGB values of the skin and camera model are at a minimum, the RGB values corresponding to the skin and camera model are stored in memory 26.

Alternatively, if the RGB error (block 236) is not reduced or minimized, the estimates of skin characteristics are determined again. That is, the skin characteristics of the equation 27 are scaled according to equation 28 (block 240). In some embodiments, the newly generated estimates of the skin characteristics may be diagonally scaled (block 242), as mentioned above. After the newly generated estimates of the skin characteristics of equation 1 are scaled, a skin and camera model is generated. The RGB values corresponding to the skin and camera model are extrapolated (block 234) and compared with the averaged RGB values of the video stream 14. The RGB error is calculated (block 236) and iteratively determined whether the RGB error is minimized by the first optimizer (block 238). In some embodiments, the process 230 of FIG. 7 is iteratively executed until the difference between the averaged RGB values from the video stream 14 and the RGB values of the skin and camera model are at a minimum.

Turning to FIG. 8, depicted is an embodiment of a process 250 executing a second stage of the model inversion method of FIG. 6, whereby the error is reduced between the pulse signal with minimum SNR and the pulse signal extracted from the skin and camera model to generate final skin characteristics. In this stage, the skin characteristics of equation 1 are estimated over time using frame by frame RGB values from the region of interest. Afterwards, an objective function provided by complex-step method and the chain rule method may be used as a second optimizer. In other words, after the difference between the averaged RGB values from the video stream 14 and the RGB values of the skin and camera model is at a minimum, the pulse signal, Pₘ(t) corresponding to the RGB of the skin and camera model is set as final. The skin characteristics of equation 1 corresponding to the pulse signal (e.g., a second representative physiological signal), Pₘ(t), of the RGB of the skin and camera model are set as final.

In more detail, the pulse signal, P(t) of a cycle, with the high SNR (e.g., computed using the MaxSNR method) (block 252) is compared with the pulse extracted, Pₘ(t), from the RGB values from the skin and camera model (block 254). It should be noted that the pulse signal, P(t) shown in FIG. 8 is different from the projection matrix, P in equation 20. The difference between P(t) and Pₘ(t) (e.g., the difference between representative physiological signal and the second representative physiological signal), hereinafter called “the pulse signal error,” is computed (block 256). In some embodiments, the pulse signal error is processed by a second optimizer (block 258) which may use equations 29 and 30 to iteratively reduce the pulse signal error. As such, if the pulse signal error is not a minimum, process 250 proceeds to scale parameters via equation 31.

$${P}_{3} = [ \begin{matrix}\frac{{f}_{blood}}{{α}_{1}} & \frac{{SO}_{2}}{{α}_{2}} & \frac{{C}_{s}}{{α}_{3}}\end{matrix} ] \qquad ( 31 )$$

where equation 31 scales only a fraction of the skin characteristics of equation 1 because, in some instances, only the skin characteristics of equation 31 are not constant. That is, in some embodiments, the skin characteristics Cₘₑₗ and Lₑₚᵢ may not vary between iterations (block 264).

After the skin characteristics have been scaled based on equation 31, in certain embodiments, the skin characteristics may be diagonally scaled (block 262). As previously mentioned, certain skin characteristics (e.g., Cₘₑₗ and Lₑₚᵢ) may be held constant (block 264) during the iteration of process 250. After the skin characteristics have been scaled (e.g., diagonally scaled), a skin and camera model is generated and the RGB values for the skin and camera model are noted (bloc 234). Furthermore, the pulse, Pₘ(t), is extracted from the RGB values for the skin and camera model (block 234), as mentioned above. The pulse signal error is again computed via equations 29 and 30.

Alternatively, if the RGB values are at a minimum, based on equations 29 and 30, the RGB values and the skin and camera model are stored in memory 26. Afterwards, in some embodiments, the skin characteristics corresponding to the stored RGB values and the skin and camera are used to generate a PPG waveform and any target skin characteristics, as mentioned above.

FIG. 9 is a flow diagram 270 depicting an embodiment of a general process, whereby final physiological parameters are generated based on a video stream 14 captured by a camera device 10. In more detail, the illustrated embodiment includes a first schematic 272 that may be displayed on display 19 and stored in memory 26 until processed via processor 24. As illustrated, a person may record a video stream 14 of an area of interest (e.g., forehead 16 or any substantially flat surface).

After recording video stream, RGB image data (process block 106 of FIG. 3) may be generated based on manufacturing specifications of the camera capturing the recorded video stream 14, as illustrated in the second schematic 274. The RGB image data may include RGB signals with respect to time. For example, as illustrated, the display 19 may include a graph 756 of red signal over time, a graph 757 of green signal overtime, a graph 758 of blue signal overtime. In some embodiments, the waveforms may be individually plotted as illustrated in the second schematic 274. In further embodiments, the waveforms may be plotted on one graph.

Furthermore, after the RGB image data is obtained, the RGB values may be spatially averaged to generate averaged RGB values, Tₘₑₐₙ=[Rₘₑₐₙ Gₘₑₐₙ Bₘₑₐₙ]^{T} (block 172 of FIG. 5). As illustrated in the third schematic 276, the averaged RGB values may be displayed on the camera device 10. In some embodiments, the PPG waveform and/or the pulse signal, P(t), generated by the MaxSNR method (process block 186 of FIG. 5) may be displayed on the display of the camera device 10.

Finally, based on the calculations discussed above, the model inversion method may be used to reduce pulse signal error to generate values of the skin characteristics. As illustrated by the fourth schematic 278, various skin characteristics and physiological parameters may be provided as final (process block 210 of FIG. 6). For example, the camera device 10 may display values for the systolic and diastolic blood pressure, in addition to the skin characteristics of equation 1, based on the calculations of the model inversion method, described in detail above with regards to FIGS. 6-8.

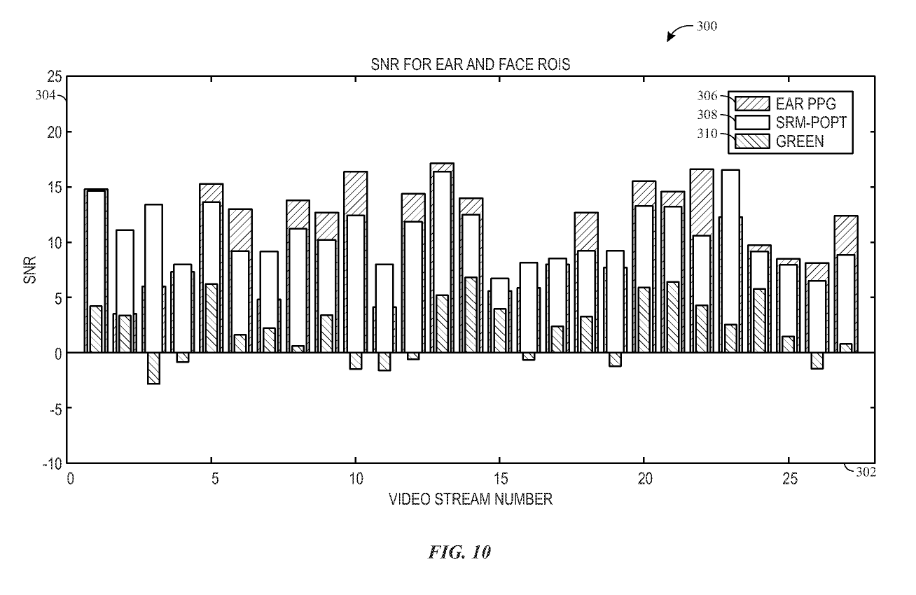

For context regarding data validation for the subject matter of this disclosure, FIG. 10 depicts an embodiment of experimental data on a bar graph 300, whereby the SNR for 27 video streams is compared. That is, the SNR was computed and plotted for traditional methods such as ear PPG 306 and green only methods 310 with frame rates described with regards to FIG. 5, (e.g., process block 176). Then the SNR was computed using the proposed method 308 (e.g., MaxSNR method). As illustrated, the bar graph 300 includes the video stream number on the horizontal axis 302 and the computed SNR on the vertical axis 304. Each of the vertical spikes correspond to each of the 27 video streams that were taken for each of the aforementioned three methods (306, 308, and 310).

The experiment involved volunteers performing various activities to vary their blood pressure (e.g., between low and high) during which the blood pressure and various other PPG retrieval methods involving electrocardiograms (ECG), finger or ear PPG (e.g., ear PPG is displayed on FIG. 10), facial and hand video (e.g., displayed as the proposed method 308), were captured at rest (e.g., baseline), and then again after lowering and elevating blood pressure. Video images captured at specified frame rates during various blood pressure conditions contain pulsatile information. Two regions of interest (ROI) were selected for each video: proximal (e.g., face) and distal (e.g., hand). These ROI were fed to an (e.g., MATLAB) algorithm that compared the methods shown in graph 300. The SNR was computed using equation 32.

$$SNR = 10 log 10 ( \frac{Psig}{Pnoise} ) \qquad ( 32 )$$

where Pnoise=Ptotal−Psig, such that band pass filter may include significant cardiac frequencies (e.g., fundamentally tuned to the pulse rate frequency, first harmonic, second harmonic etc). In the results shown on FIG. 10, the fundamental and first harmonic frequencies were used. Ptotal is the power of the original signal obtained prior to multi-band filtering.

Furthermore, a comparison of the methods for in terms of the signal to noise ratio, for 27 videos, are shown below. As illustrated, the proposed method 308 is compared closely with the PPG signals, which are not subject to issues of motion. In addition, the mean and standard deviation across all the videos are listed FIG. 11. In FIG. 11, the SNR corresponding to the best of the existing methods (e.g., FIG. 5 process blocks 172-180) are compared with the proposed method 308 to illustrate the quality of the pulse signal as well as the potential to reach the PPG quality.

FIG. 11 depicts an embodiment of a table 320 illustrating data comparison between the existing method of generating PPG and the proposed method of FIG. 3, based on the experimental data of FIG. 10. As depicted, the means 322 and the standard deviations (STD) corresponding to the ear PPG 306, the green only methods 310, and the proposed method 308 (e.g., MaxSNR method).

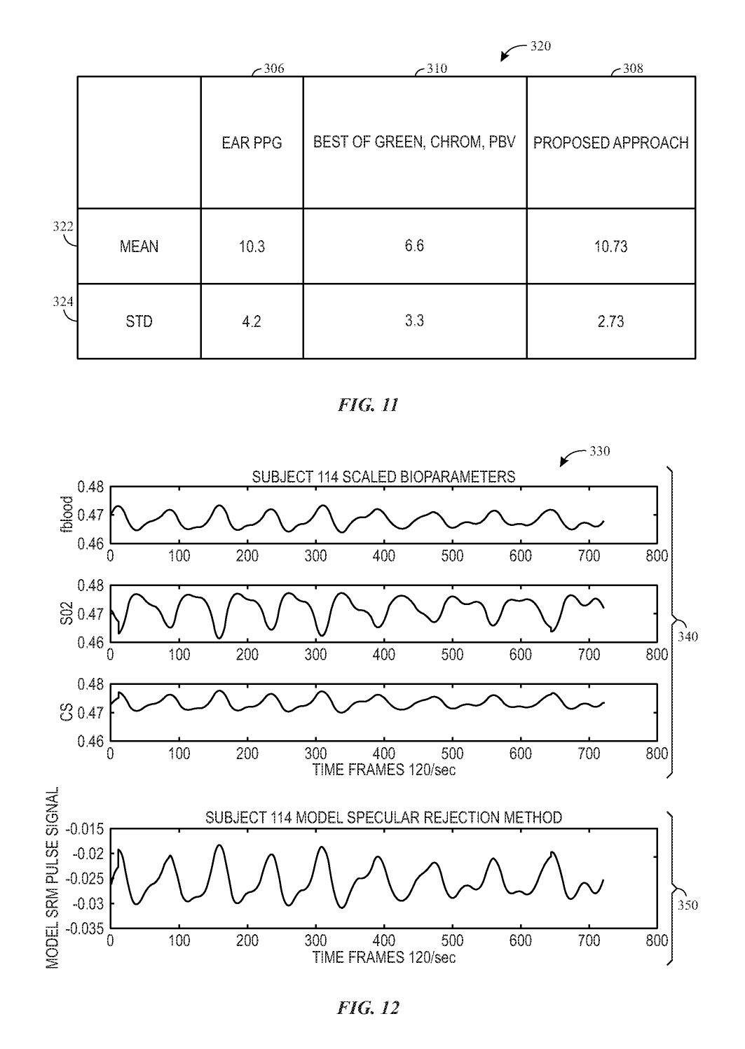

FIG. 12 depicts a set of plots 330 of the signal 350 retrieved from scaled skin characteristics 340, utilizing the MaxSNR method of FIG. 5. Furthermore, as previously mentioned, the plot of scaled skin characteristics may include only a subset of the skin characteristics of equation 1 because certain skin characteristics (e.g., Cₘₑₗ and Lₑₚᵢ) may be held constant (block 264).

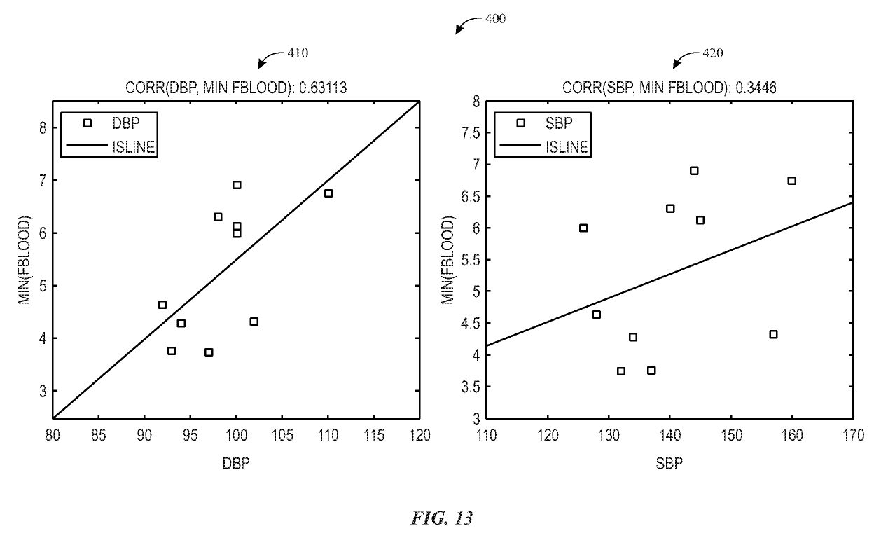

FIG. 13 depicts a plot set 400 of evaluated correlation of time averaged blood concentration parameter to systolic blood pressure (SBP) in plot 420 and diastolic blood pressure (DBP) 410. More specifically, FIG. 13 shows that the correlation between DBP vs. ƒ_{blood} was 0.63 and the correlation between SBP vs. ƒ_{blood} was 0.34.

Technical effects of the disclosure include generating a PPG waveform via a camera device (e.g., multispectral/RGB camera) as opposed to traditional contact-based PPG devices. The disclosed subject matter uses a model-based approach to extract physiological parameters from skin characteristics, such that the effects of light intensity, variations in camera, effects of motion, effects of specular light reflection, etc. are reduced to improve the signal-to-noise (SNR). After maximizing the SNR, the pulse signal (e.g., representative physiological signal) with the improved SNR is compared to the pulse signal of estimate skin characteristics (e.g., the second representative physiological signal) until the error between the two pulse signals is reduced. The skin characteristics corresponding to the pulse signal with the reduced error are determined as final, and may be displayed on the camera device, thereby providing a portal approach to determining physiological parameters indicative of a person's health.

This written description uses examples to disclose the claimed subject matter, including the best mode, and also to enable any person skilled in the art to practice the claimed subject matter, including making and using any devices or systems and performing any incorporated methods. The patentable scope of the subject matter is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A system, comprising:
an imaging device configured to capture multichannel image data from a region of interest on a patient;
one or more processors; and
memory storing instructions, wherein the instructions are configured to cause the one or more processors to:
receive the multichannel image data from the imaging device, wherein the multichannel image data comprises an image signal representative of plethysmographic waveform data for the region of interest and specular noise in the multichannel image data;
generate a projection matrix associated with the multichannel image data;
iterate values of the projection matrix to suppress the specular noise to generate a representative physiological signal, wherein the representative physiological signal has an improved signal-to-noise ratio relative to the image signal and wherein the representative physiological signal is a representative plethysmographic waveform;
calculate one or more physiological parameters using the representative physiological signal; and
output the one or more physiological parameters on a display.

2. The system of claim 1, wherein the instructions configured to cause the one or more processors to receive the multichannel image data comprise temporally averaging and spatially averaging the multichannel image data.

3. The system of claim 1, wherein the imaging device comprises a red, green, and blue (RGB) camera, a multispectral camera, a hyperspectral camera, a four-channel RGB and near infrared camera, a multichannel near infrared camera, a multichannel short wave infrared camera, or any combination thereof.

4. The system of claim 1, wherein the instructions configured to cause the one or more processors to generate a representative physiological signal comprise iteratively determining the projection matrix that reduces the signal-to-noise ratio of the representative physiological signal.

5. The system of claim 1, wherein the one or more physiological parameters comprise a blood oxygen saturation, heart rate variability, heart rate, blood pressure, or any combination thereof.

6. The system of claim 1, wherein one or more physiological parameters are determined by modeling a plurality of skin characteristics comprising the epidermis layer, the melanin concentration of skin, the volume fraction of a dermis layer, and the scattering coefficient of the dermis and the epidermis layer.

7. The system of claim 6, wherein the instructions configured to cause the one or more processors to calculate one or more physiological parameters using the representative physiological signal comprise iteratively varying at least one of the skin characteristics to remove a difference between the image signal and the representative physiological signal.

8. The system of claim 7, wherein the instructions configured to cause the one or more processors to calculate one or more physiological parameters using the representative physiological signal comprise minimizing the difference between the image signal and the representative physiological signal.

9. The system of claim 1, wherein the imaging device, the processor, and the memory are housed within a personal mobile device.

10. The system of claim 1, wherein one or more channels in the multichannel image data are normalized, wherein normalizing the one or more channels eliminates mean and higher order variations in intensity data of the multichannel image data, specular data of the multichannel image data, and pulse data of the multichannel image data.

11. The system of claim 1, wherein the region of interest comprises a substantially flat surface of skin associated with the patient.

12. A method, comprising:
acquiring multichannel image data using an imaging device from a region of interest on a patient, wherein the multichannel image data comprises an image signal representative of plethysmographic waveform data for the region of interest and specular noise in the multichannel image data, wherein the multichannel image data comprises intensity data, specular data, and pulse data;
generating a projection matrix of the multichannel image data;
iterating values of the projection matrix to remove the specular noise to generate a representative physiological signal, wherein the representative physiological signal has an improved signal-to-noise ratio relative to the image signal and wherein the representative physiological signal is a representative plethysmographic waveform;
calculating one or more physiological parameters using the representative physiological signal; and
displaying the one or more physiological parameters.

13. The method of claim 12, wherein one or more channels in the multichannel image data are normalized, wherein normalizing the one or more channels eliminates mean and higher order variations in intensity data associated with the multichannel image data, specular data associated with the multichannel image data, and pulse data associated with the multichannel image data.

14. The method of claim 13, wherein normalizing the one or more channels comprises generating a diagonal matrix comprising values between zero and 1, wherein the values are associated with the multichannel image data.

15. The method of claim 12, wherein the imaging device comprises a red, green, and blue (RGB) camera, a multispectral camera, a hyperspectral camera, a four-channel RGB and Near Infrared camera, a multichannel near infrared camera, a multichannel short wave infrared camera, or any combination thereof.

16. The method of claim 12, wherein calculating one or more physiological parameters comprises iteratively minimizing the difference between the image signal and the representative physiological signal.

17. A personal mobile device system, comprising:
an imaging device configured to capture image data over time from a region of interest on a patient, wherein the image data comprises an image signal representative of plethysmographic waveform data for the region of interest and noise;
one or more processors; and
a memory storing instructions, wherein the instructions are configured to cause the one or more processors to:
generate a projection matrix of the image data, wherein the projection matrix is based on a number of spectral components in the image data;
iterate values of the projection matrix to suppress the noise representative of the specular reflection to generate a representative physiological signal, wherein the representative physiological signal has an improved signal-to-noise ratio relative to the image signal and wherein the representative physiological signal is a first representative plethysmographic waveform;
fit a second representative physiological signal to the representative physiological signal, wherein the second representative physiological signal is generated based on a model of skin characteristics of the patient; and
display the one or more physiological parameters.

18. The mobile device system of claim 17, wherein one or more channels in the image data are normalized, wherein normalizing the one or more channels eliminates mean and higher order variations in intensity data associated with the image data, specular data associated with the image data, and pulse data associated with the image data.

19. The mobile device system of claim 17, wherein the memory storing instructions configured to cause the one or more processors to fit the second representative physiological signal to the representative physiological signal comprises fitting a second plethysmographic waveform signal associated with the second representative physiological signal with the first plethysmographic waveform.

20. The mobile device system of claim 17 is configured to be communicatively coupled to an external computing device, wherein the external computing device is configured to iterate values of the projection matrix and fit the second representative physiological signal to the representative physiological signal.
